(19) United States
(12) Patent Application Publication
(10) Pub. No.: US 2025/0288224 A1
(43) Pub. Date: Sep. 18, 2025

(54) METHODS AND SYSTEMS FOR MEDICATION DETERMINATION AND MANAGEMENT

(71) Applicant: Abbott Diabetes Care Inc., Alameda, CA (US)

(72) Inventors: Yue RUAN, Oxford (GB); Erwin Satrya BUDIMAN, Fremont, CA (US); Matthew T. NOVAK, Somerville, CA (US); Gary A. HAYTER, Oakland, CA (US); Marc TAUB, Danville, CA (US); Sudhir Mahakali, Tustin, CA (US)

(21) Appl. No.: 19/079,997

(22) Filed: Mar. 14, 2025

Related U.S. Application Data
(60) Provisional application No. 63/565,753, filed on Mar. 15, 2024.

(51) Int. Cl. A61B 5/145; G16H 20/10

(52) U.S. Cl. CPC A61B 5/14532; G16H 20/10

(57) ABSTRACT

Systems and methods of optimizing glucose medication dosages are based on modeling a patient's glucose lowering response using historical glucose data. The model is actively updated to account for changes in the patient's medication treatments and physiological conditions based on real-time or near real-time glucose measurements from the patient. The system can analyze various dosages of insulin based on the patient's current therapy and the model to determine an optimized dosage. Also disclosed is a system and method for retrieving a patient's current medication therapies from medical records using an automated system. Also disclosed are methods of implementing remotely-updatable limitations for medication optimization algorithms.

### CROSS-REFERENCE TO RELATED APPLICATIONS

This application claims the benefit of U.S. Provisional Application No. 63/565,753, filed Mar. 15, 2024, which is incorporated herein by reference in its entirety.

### FIELD

The subject matter of this disclosure generally relates to systems, devices, and methods for management of glucose disorders. More specifically, this disclosure relates to analyzing a patient's glucose data to determine a suitable medication dosage, and systems and methods for managing prescribed medications.

### BACKGROUND

Glucose disorders occur when a patient's blood glucose levels drop below or rise above the normal blood glucose range. There are several known therapies that can be initiated to address these types of disorders. Typically, treatment is initiated in consultation with a health care professional after a patient presents with symptoms. A blood glucose reading is usually taken to confirm the patient's blood glucose level prior to treatment.

The introduction of personal continuous glucose monitors (“CGM”) has increased the frequency of glucose data available to a patient and their health care professional. Clinical studies in hospital settings have shown that computerized algorithms can improve patient outcomes by allowing a tighter control over blood glucose levels by analyzing data from hospital-based CGMs. This disclosure details systems and methods of using data received from a CGM to analyze the blood glucose levels of a patient and determine various metrics for improving treatment dosage determination. Also included here are systems and methods for monitoring the medication for the various treatments a patient is undergoing.

### BRIEF SUMMARY OF THE INVENTION

In an embodiment a method of determining an optimized medication dosage for a glucose disorder, includes receiving, by one or more processors, glucose data from a continuous glucose monitor worn by a patient; receiving, by the one or more processors, medication data for the patient; selecting, by the one or more processors, a set of alternate medication dosages based on the medication data; determining a predicted glucose response for each medication dosage of the set of alternate medication dosages; analyzing each predicted glucose response to determine an optimized medication dosage of the set of medication dosages; and outputting the recommended medication dosage by a display device in communication with the one or more processors.

In a further embodiment, the glucose data includes glucose measurements recorded on a plurality of days.

In a further embodiment the predicted glucose response for each medication dosage is determined based in part on the glucose data and a glucose lowering curve.

In a further embodiment the updating includes updating an insulin sensitivity for the patient using the glucose data and the medication data.

In a further embodiment the updating includes updating a peak insulin response for the patient using the glucose data and the medication data.

In a further embodiment the medication data includes a treatment dosage and a treatment time corresponding to when the treatment dosage was administered, and wherein updating the glucose lowering curve further includes determining an insulin sensitivity of the patient by analyzing a response in glucose measurements in the glucose data after the treatment time.

In a further embodiment the glucose data includes glucose measurements recorded on a plurality of days, wherein the insulin sensitivity is one of a plurality of insulin sensitivities, each insulin sensitivity corresponding to each day of the plurality of days, and wherein updating the glucose lower curve further includes taking an average of the plurality of insulin sensitivities.

In a further embodiment, the selecting the set of alternate medication dosages includes determining a new dosage by modifying an existing dosage from the medication data by at least one of increasing or decreasing the existing dosage.

In a further embodiment analyzing each predicted glucose response further includes calculating a metric for each predicted glucose response, the metric being at least one of median glucose versus a target glucose, time in a predetermined glucose range, time in hypoglycemia, number of hypoglycemic events, or glycemic variability.

In a further embodiment the method includes receiving a limitation on a medication dosage from a remote computing device, wherein selecting the set of alternate medication dosages is based in part on the limitation.

In a further embodiment the limitation includes one or more of a maximum dosage for an individual medication; a maximum total dosage of medication for a predetermined time period, and a maximum dosage for an individual medication dosage related to a second medication dosage.

In a further embodiment the receiving medication data further includes receiving account information for an account associated with the patient receiving medication, the account having information related to the medication data; and using the account information to access the account to retrieve the information related to the medication data.

In a further embodiment receiving account information includes acquiring the account information from an object using a reader device.

In a further embodiment the reader device includes a camera that is used to acquire the account information from the object.

In a further embodiment the account is an electronic medical record, and wherein the information related to the medication data is a second account information for a second account that includes the medication data, the method further using the second account information to access the second account to retrieve the medication data.

In a further embodiment the information related to the medication data is incorporated into the recommended medication dosage.

In a further embodiment the medication data is received from an insulin delivery device.

In a further embodiment the medication data is received from a user input.

In a further embodiment the medication data includes an insulin data.

In an embodiment, a method of initializing an application including a medication dosage guidance algorithm on a display device of a patient, includes transmitting from a computing device of a health care provider, to a pharmacy computing device, an order for the application; receiving, from the pharmacy information system, a code for initializing the application at an electronic medical record of a health care professional using an authentication system; displaying the code using a display device in communication with the electronic medical record; and receiving the code by the display device of the patient to initialize the application.

In a further embodiment the receiving the code includes receiving an inquiry at an authentication system for the code from the pharmacy; and transmitting the code to the pharmacy in response to the inquiry.

In a further embodiment receiving the code at the display device includes scanning the code by a reader device of the display device.

In a further embodiment the authentication system receives an identifier of the HCP and returns an authentication code based in part on the identifier of the HCP.

In an embodiment, a system for determining an optimized therapy for a glucose disorder, includes a continuous glucose monitor, including: a glucose sensor including a first portion configured to be arranged above the skin and a second portion configured to be arranged below the skin and in contact with interstitial fluid to sense glucose within the interstitial fluid, and sensor electronics coupled to the glucose sensor and including a memory, communication circuitry, and a processor coupled to the memory and communication circuitry; and a computing device including a second processor, a second memory, and a display, wherein the computing device is in communication with the continuous glucose monitor, wherein the second memory stores instructions that when executed by the second processor cause the second processor to: receive glucose data from the continuous glucose monitor; receive medication therapy data for the patient at the computing device; select a set of medication therapy dosages based on the medication therapy data; determine a predicted glucose response for each medication therapy dosage of the set of medication therapy dosages; analyze each predicted glucose response to determine an optimized medication therapy dosage of the set of medication therapy dosages; and output the optimized medication therapy dosage on the display of the computing device.

In a further embodiment the glucose data includes glucose measurements recorded on a plurality of days.

In a further embodiment the predicted glucose response for each medication therapy dosage is determined based in part on the glucose data and a glucose lowering curve.

In a further embodiment the updating includes instructions that further cause the second processor to update an insulin sensitivity for the patient using the glucose data and the medication therapy data.

In a further embodiment the updating includes instructions that further cause the second processor to update a peak insulin response for the patient using the glucose data and the medication therapy data.

In a further embodiment the medication therapy data includes a treatment dosage and a treatment time corresponding to when the treatment dosage was administered, and wherein the instructions to update the glucose lowering curve cause the second processor to determine an insulin sensitivity of the patient by analyzing a response in glucose measurements in the glucose data after the treatment time.

In a further embodiment the glucose data includes glucose measurements recorded on a plurality of days, wherein the insulin sensitivity is one of a plurality of insulin sensitivities, each insulin sensitivity corresponding to each day of the plurality of days, and wherein the instructions to update the glucose lowering curve further cause the second processor to take an average of the plurality of insulin sensitivities.

In a further embodiment the instructions to select the set of medication therapy dosages further cause the second processor to determine a new dosage by modifying an existing dosage from the medication therapy data by at least one of increasing or decreasing the existing dosage.

In a further embodiment the instructions to analyze each glucose response further cause the second processor to calculate a metric for each glucose response, the metric being at least one of median glucose versus a target glucose, time in a predetermined glucose range, time in hypoglycemia, number of hypoglycemic events, and glycemic variability.

In a further embodiment the instructions further cause the second processor to receive a limitation on a medication therapy dosage from a remote computing device, wherein the selecting a set of medication therapy dosages is based in part on the limitation.

In a further embodiment the limitation is selected from the group consisting of a maximum dosage for an individual medication; a maximum total dosage of medication for a predetermined time period, and a maximum dosage for an individual medication dosage related to a second medication dosage.

In a further embodiment the instructions to receive medication therapy data further cause the second processor to: receive account information for an account associated with the patient receiving medication therapy, the account having information related to the medication therapy data; and use the account information to access the account to retrieve the information related to the medication therapy data.

In a further embodiment the system includes a reader device, wherein the instructions to receive account information further cause the second processor to use the reader device to acquire the account information from an object.

In a further embodiment the reader device includes a camera that is used to acquire the account information from the object.

In a further embodiment the account is an electronic medical record, and wherein the information related to the medication therapy data is a second account information for a second account that includes the medication therapy data, the instructions further including instructions to cause the second processor to use the second account information to access the second account to retrieve the medication therapy data.

In a further embodiment the information related to the medication therapy data is incorporated into the recommended medication dosage.

In a further embodiment the medication therapy data is received from an insulin delivery device.

In a further embodiment the medication therapy data is received from a user input.

In a further embodiment the medication therapy data include an insulin medication therapy.

In an embodiment, a system for determining an optimized therapy for a glucose disorder, includes: a continuous glucose monitor, including: a glucose sensor including a first portion configured to be arranged above the skin and a second portion configured to be arranged below the skin and in contact with interstitial fluid to sense glucose within the interstitial fluid, and sensor electronics coupled to the glucose sensor and including a memory, communication circuitry, and a processor coupled to the memory and communication circuitry; and a computing device including a second processor, a second memory, and a display, wherein the computing device is in communication with the continuous glucose monitor, wherein the second memory stores instructions that when executed by the second processor cause the second processor to: transmit from a computing device of a health care provider, to a pharmacy computing device, an order for the application; receive, from the pharmacy information system, a code for initializing the application at an electronic medical record of a health care professional using an authentication system; display the code using a display device in communication with the electronic medical record; and receive the code by the display device of the patient to initialize the application.

In a further embodiment the receiving the code further includes instructions that further cause the second processor to: receive an inquiry at an authentication system for the code from the pharmacy; and transmit the code to the pharmacy in response to the inquiry.

In a further embodiment receiving the code further includes instructions that cause the second processor to scan the code by a reader device of the display device.

In an embodiment, a system for determining an optimized therapy for a glucose disorder includes: a continuous glucose monitor, including: a glucose sensor including a first portion configured to be arranged above the skin and a second portion configured to be arranged below the skin and in contact with interstitial fluid to sense glucose within the interstitial fluid, and sensor electronics coupled to the glucose sensor and including a memory, communication circuitry, and a processor coupled to the memory and communication circuitry; and a computing device including a second processor, a second memory, and a display, wherein the computing device is in communication with the continuous glucose monitor, wherein the second memory stores instructions that when executed by the second processor cause the second processor to: generate a unique code for a health care provider based on an inquiry from the health care provider; receive the unique code at the application; and authenticate the unique code to initialize the application.

Systems and methods of optimizing glucose medication dosages may be based on modeling a patient's glucose lowering response using historical glucose data. The model may be actively updated to account for changes in the patient's medication treatments and physiological conditions based on real-time or near real-time glucose measurements from the patient. The system can analyze various dosages of insulin based on the patient's current therapy and the model to determine an optimized dosage. Also disclosed is a system and method for retrieving a patient's current medication therapies from medical records using an automated system. Also disclosed are methods of implementing remotely-updatable limitations for medication optimization algorithms. Certain aspects of the disclosure have other steps or elements in addition to or in place of those mentioned above. The steps or elements will become apparent to those skilled in the art from a reading of the following detailed description when taken with reference to the accompanying drawings.

### BRIEF DESCRIPTION OF THE DRAWINGS/FIGURES

The accompanying drawings, which are incorporated herein and form a part of the specification, illustrate the present disclosure and, together with the description, further serve to explain the principles thereof and to enable a person skilled in the pertinent art to make and use the same.

FIG. 1 is a system overview of a sensor applicator, reader device, monitoring system, network, and remote system according to an embodiment.

FIG. 2A is a block diagram depicting an example embodiment of a reader device according to an embodiment.

FIGS. 2B and 2C are block diagrams depicting example embodiments of sensor control devices according to embodiments.

FIG. 3 is a perspective view of an analyte sensor according to an embodiment.

FIG. 4 is a system diagram of a method of optimizing medication therapies according to embodiments.

FIG. 5 is an example of a glucose lowering curve according to embodiments.

FIG. 6 is a flow chart of a method of optimizing medication dosages according to embodiments.

FIG. 7 is a system diagram of a method of accessing user therapy information according to embodiments.

FIG. 8 is a system diagram of a system for accessing user therapy information according to embodiments

FIG. 9 is a system diagram of a method of remotely updating limitations for an algorithm according to embodiments.

FIG. 10 is a flow diagram of a method for initializing an application having medication dosage algorithm according to embodiments.

FIG. 11 is a flow diagram of a method for initializing an application having medication dosage algorithm according to embodiments.

In the drawings, like reference numbers generally indicate identical or similar elements. Additionally, generally, the left-most digit(s) of a reference number identifies the drawing in which the reference number first appears.

### DETAILED DESCRIPTION

Reference will now be made in detail to representative embodiments illustrated in the accompanying drawings. References to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such a feature, structure, or characteristic in connection with other embodiments whether or not explicitly described.

Glucose levels in the human body fluctuate throughout the day based on several different factors. One of the most significant factors is food intake, which results in a post-meal rise in blood glucose levels. Conversely, fasting usually results in a drop in blood glucose levels. This variability is not constant across patients. For example, different patients can have different variabilities, and can have blood glucose levels that react to meals and fasting differently. This means that a single glucose measurement taken at a health care professional's office may not be indicative of a patient's typical blood glucose levels, which can result in non-optimized therapy. Further, a patient that has begun a therapy typically visits their health care professional's office every 3-6 months, or even less frequently, to check their glucose reading and adjust their medication dosage as needed. This can result in therapy that is not tailored to the patient's most recent glucose control needs for one or both of these factors. Thus, there is a need for systems and methods for optimizing a patient's medication dosage(s).

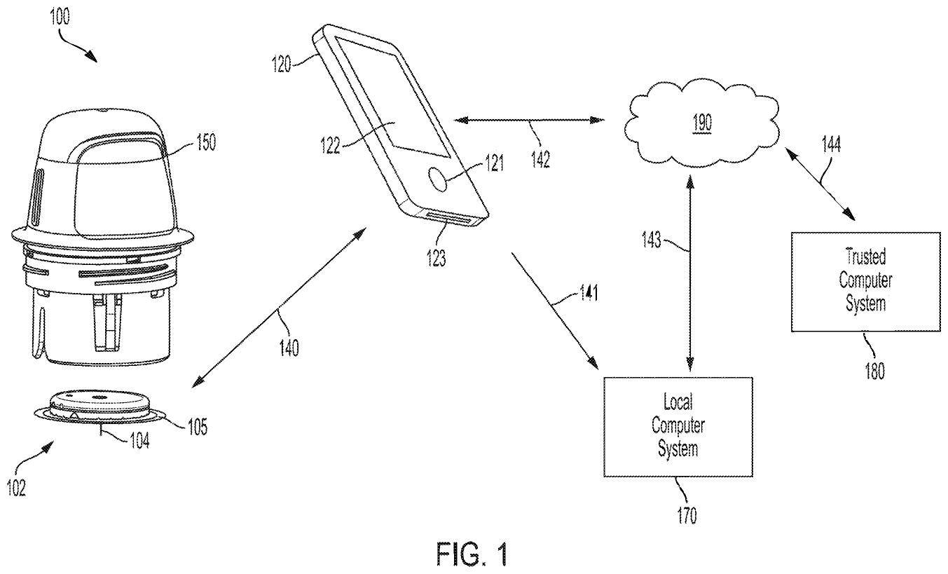

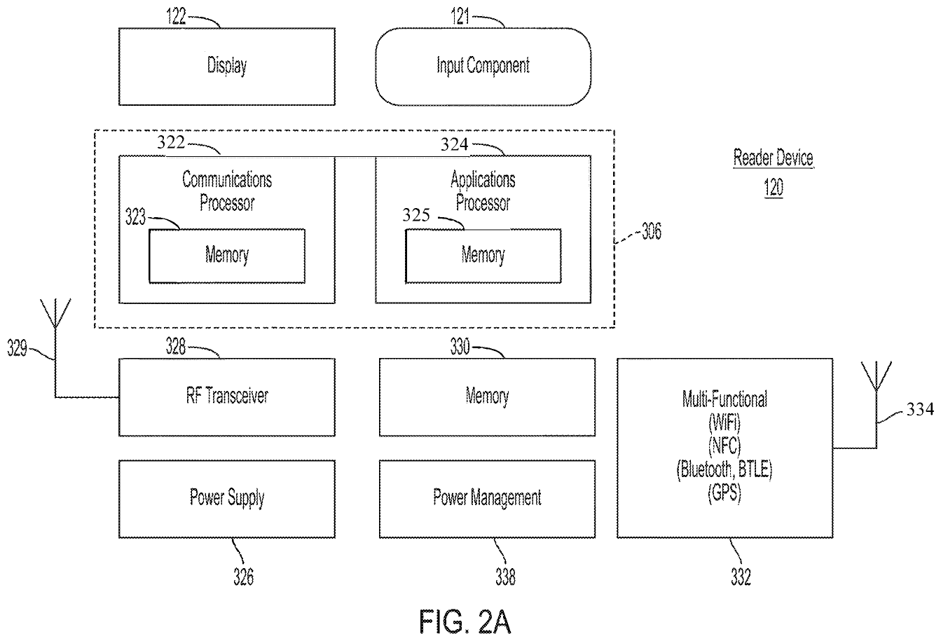

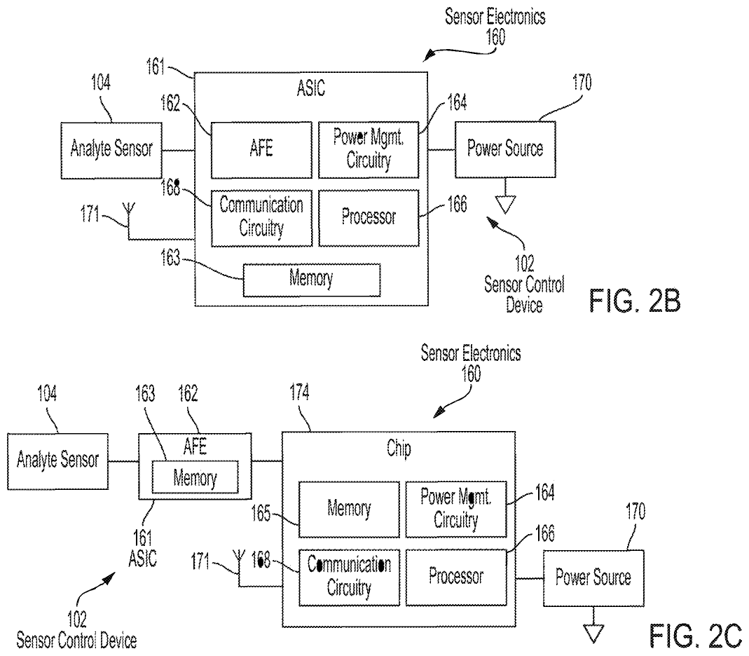

FIG. 1 is a conceptual diagram depicting an example embodiment of a continuous glucose monitor 100 that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can monitor applications installed in memory on reader device 120 using display 122 and input 121, and the device battery can be recharged using power port 123. While only one reader device 120 is shown, sensor control device 102 can communicate with multiple reader devices 120. Each of the reader devices 120 can communicate and share data with one another. More details about reader device 120 are set forth with respect to FIG. 2A below.

Reader device 120 can communicate with local computer system 170 via a communication path 141 using a wired or wireless communication protocol. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by a wired or wireless communication protocol as described previously. Network 190 can be any of a number of networks, such as private networks and public networks, local area or wide area networks, and so forth. A trusted computer system 180 can include a server and can provide authentication services and secured data storage and can communicate via communications path 144 with network 190 by wired or wireless technique.

FIG. 2A is a block diagram depicting an example embodiment of a reader device 120 configured as a smartphone. Here, reader device 120 can include a display 122, input component 121, and a processing core 306 including a communications processor 322 coupled with memory 323 and an applications processor 324 coupled with memory 325. Also included can be separate memory 330, RF transceiver 328 with antenna 329, and power supply 326 with power management module 338. Further, reader device 120 can also include a multi-functional transceiver 332 which can communicate over Wi-Fi, NFC, Bluetooth, BTLE, and GPS with an antenna 334. As understood by one of skill in the art, these components are electrically and communicatively coupled in a manner to make a functional device.

FIGS. 2B and 2C are block diagrams depicting example embodiments of sensor control devices 102 having analyte sensors 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 170, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, including the algorithms described in detail below. This data can then be provided to communication circuitry 168 for sending, by way of antenna 171, to reader device 120 (not shown), for example, where minimal further processing is needed by the resident software application to display the data.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 includes memory 163 and chip 174 includes memory 165, which can be isolated or distributed within. In one example embodiment, AFE 162 is combined with power management circuitry 164 and processor 166 on one chip, while communication circuitry 168 is on a separate chip, In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

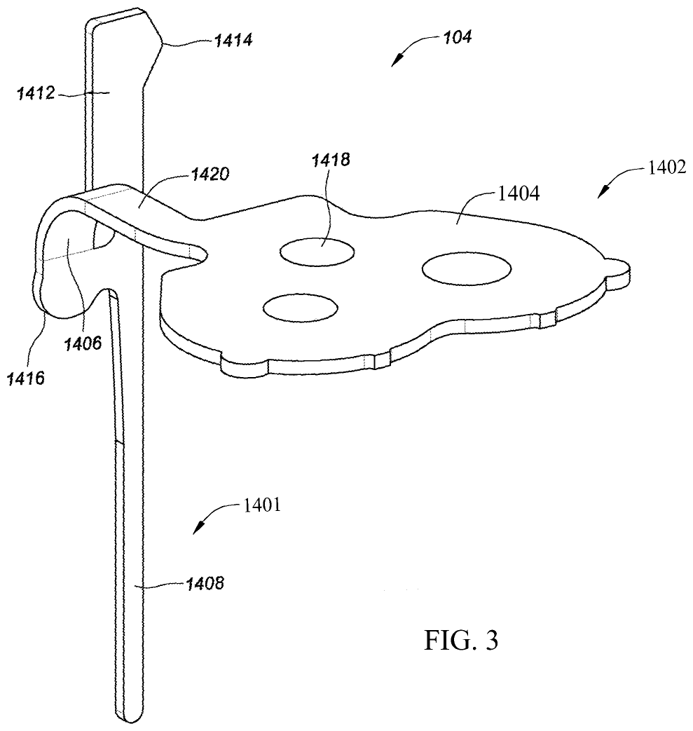

FIG. 3 is a perspective view depicting an example embodiment of an analyte sensor 104. Sensor control device 102 may include an analyte sensor 104 that is a transcutaneous sensor having an in-vivo portion 1401 and an ex-vivo portion 1402. In-vivo portion 1401 is the portion that is inserted into the patient. For example, in-vivo portion 1401 may be inserted into the skin of the patient and be placed into contact with a bodily fluid, such as interstitial fluid. However, in alternate embodiments, analyte sensor may be in contact with other bodily fluids, such as blood. Sensor tail 1408 can be the portion of sensor 104 that resides under a user's skin after insertion. Sensor tail 1408 may include one or more active sensing areas for detecting a presence of an analyte in the bodily fluid. The active sensing area may include one or more electrodes, such as a working electrode, counter electrode and/or reference electrode. A reagent for detection of the analyte may be disposed on one or more of the electrodes, such as on a working electrode, and the reagent may include an analyte responsive reagent. A membrane on tail 1408 can cover an active analyte sensing element of analyte sensor 104.

Ex-vivo portion 1402 of analyte sensor 104 remains outside of the patient and is the portion of sensor 104 that mechanically and electrically interfaces with other elements, such as sensor electronics 160. A neck 1406 can be a zone which allows folding of the sensor, for example ninety degrees. A flag 1404 can contain contacts and a sealing surface. A biasing tower 1412 can be a tab that biases the tail 1408 for mechanical connection. A bias fulcrum 1414 can be an offshoot of biasing tower 1412 that contacts an inner surface of a needle to bias a tail into a slot. A bias adjuster 1416 can reduce a localized bending of a tail connection and prevent sensor trace damage. Contacts 1418 can electrically couple the active portion of sensor 104 to suitable contacts for electrical connection to sensor electronics 160. A service loop 1420 can translate an electrical path from a vertical direction ninety degrees to flag 1404.

The components discussed above provide data monitoring and processing capabilities that can be used to determine an improved medication dosage for a patient. Embodiments of a therapy algorithm 200 discussed below use the data and processing capabilities to produce treatment therapies tailored to the relevant patient.

A display device, such as reader device 120 or local computer system 170, in communication with the analyte sensor may include an analyte monitoring software application for displaying analyte data to the user, such as a current analyte level, trend, graph of analyte levels over time, alerts, such as high and low analyte level alerts, and other information regarding operation of the analyte sensor. The display device may also include a therapy algorithm 200 for determining a recommended dose of medication for a user to administer. The therapy algorithm 200 may be a stand-alone software application separate from a user's analyte sensor. In some embodiments, therapy algorithm 200 may be integrated in an analyte monitoring software application, and may be a sub-module integrated into the analyte monitoring software application and embedded in the user interface. The display device may be configured to receive user input of information for use in therapy algorithm, such as to enter one or more doses of medication administered by the user. In alternate embodiments, the display device and therapy algorithm may be in communication with a medication delivery device for receiving the medication doses administered, such as an automated insulin delivery device, such as a pump, a pen, or the like, or from a medication dose monitoring device, such as a smart pen cap applied to an injection pen.

In some embodiments, a therapy algorithm is configured to predict a user's glucose response for a plurality of alternate medication dosages and to recommend a medication dose based on the glucose response for the plurality of alternate medication dosages. The predicted glucose responses may be based on a mathematical model and the user's historic glucose and medication data. In some embodiments, a glucose response curve for a fixed amount of a medication is used to predict the glucose response. One or more glucose metrics may be determined for each predicted glucose response, and the recommended dosage may be based on the one or more glucose metrics.

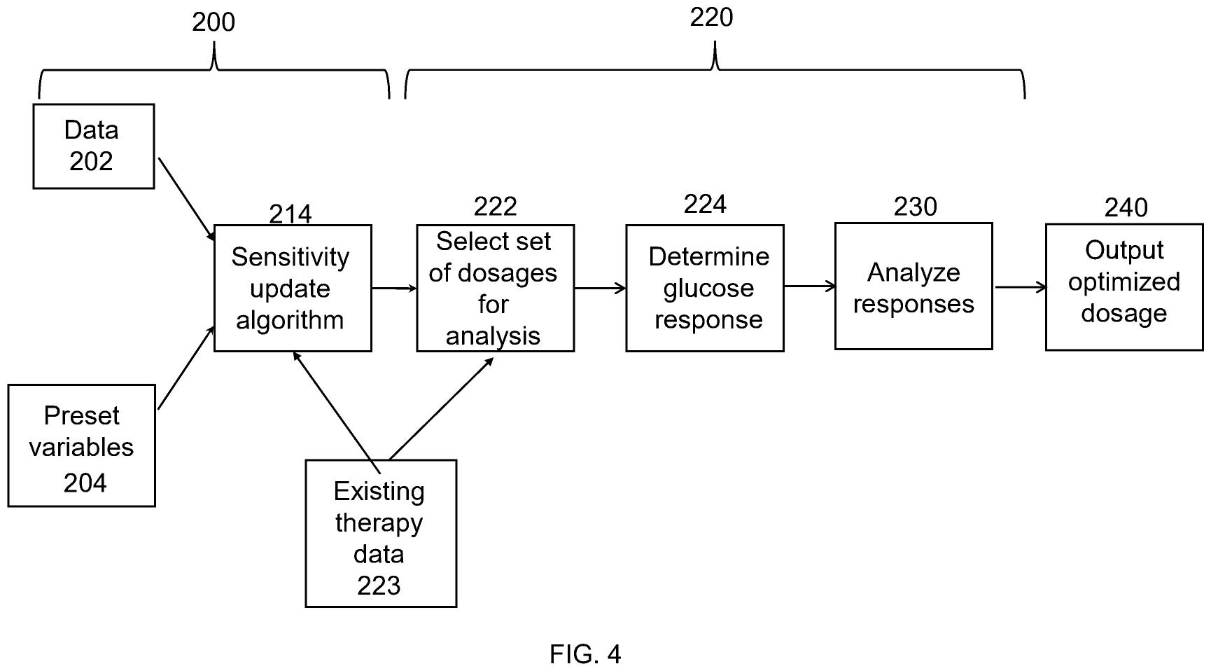

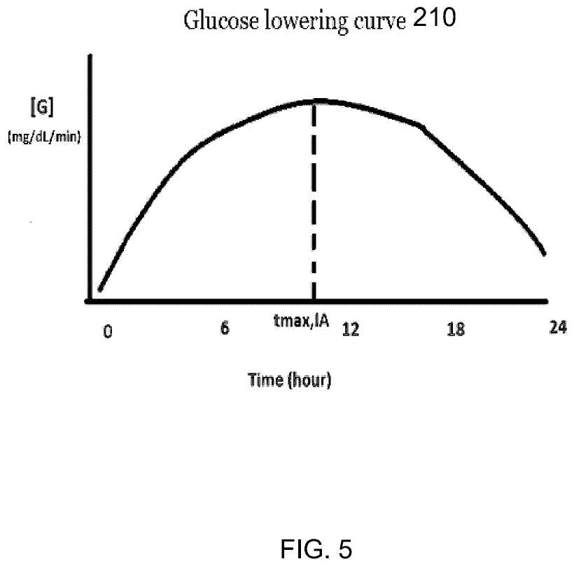

A shown in FIGS. 4 and 5, in an embodiment, therapy algorithm 200 functions to determine a glucose lowering curve 210 (FIG. 5) for a patient based on data 202 from continuous glucose monitor 100. An example glucose lowering curve 210 is shown in FIG. 5. The example of FIG. 5 illustrates the glucose level reaction to 1 U of long-acting insulin glargine. The total area under the curve represents the level of glucose concentration the 1 U dose was able to reduce.

Glucose lowering curve 210 can be defined by one or more mathematical models that define the glucose levels of a patient in response to a medication dosage (e.g., insulin). In some embodiments, glucose lowering curve 210 may be defined by a two compartment insulin action model as follows:

$$\frac{d {i}_{1} ( t )}{d t} = - \frac{1}{{t}_{max}} {i}_{1} ( t ) + {δ}_{{t}_{j}} ( t ) {D}_{Δ} ( {t}_{j} ) \qquad ( 1 )$$

$$\frac{d {i}_{2} ( t )}{d t} = \frac{1}{{t}_{max}} {i}_{1} ( t ) - \frac{1}{{t}_{max}} {i}_{2} ( t ) \qquad ( 2 )$$

$$l ( t ) = {S}_{I} ( t ) \frac{1}{{t}_{max}} {i}_{2} ( t ) \qquad ( 3 )$$

where i₁(t) and i₂(t) are the amounts of insulin in the two insulin compartments (U), I(t) is the glucose lowering curve (mg/dL per U per min), DΔ(tⱼ) denotes the difference in insulin dose amount between “original insulin dose” and an alternative insulin dose at time tⱼ (U), tₘₐₓ is the time-to-peak of insulin action (min), and S_{I} (mg/dL per U) is insulin sensitivity. The strength of the model is its simplicity as the model does not attempt to describe various physiological processes that contribute to glucose regulation, such as gut absorption, endogenous insulin secretion for people with type 2 diabetes, and insulin action at various tissues.

Glucose lowering curve 210 includes two parameters tₘₐₓ and S_{I}, where tₘₐₓ (min) represents the time-to-peak of insulin action, and insulin sensitivity S_{I} (mg/dL per U) represents the glucose-lowering effect per unit of insulin. As part of initialization on a display device, therapy algorithm 200 estimates these parameters of glucose lowering curve 210 by retrieving certain data from a memory connected to the display device. An initial estimation of S_{I} can be based on one or more preset variables 204 stored on the memory to estimate the patient's response. For example, assuming someone has a 40 U/day total insulin daily dose, the initially estimated S_{I} for that person can be estimated by dividing the dose into preset variable 204 that is set to the number 1700, which results in an S_{I} of 42.5 mg/dL per U. In some embodiments, population data for users of the medication may be used to determine preset variables 204. As will be explained below, these values may be updated over time by the therapy algorithm based on the user's data as more data is collected for the particular user.

Therapy algorithm 200 updates these parameters by reviewing historical data 202 received from a continuous glucose monitor 100 worn by the patient. As discussed above, data 202 can be received in various ways, including automatically via a suitable data connection, or manually via input from a user. For example, the insulin sensitivity parameter S_{I} can be updated by therapy algorithm 200 by matching up actual historic glucose measurements with known insulin dosing. As seen in FIG. 4, a sensitivity update algorithm 214 functions by pairing historic glucose readings from continuous glucose monitor 100 with a known dosage of insulin for a certain time period. For example, if a patient indicates that they received a dose of 40 U of insulin in a given day, update algorithm 214 retrieves the glucose data corresponding to that time period and then analyzes the glucose data to determine the resulting insulin sensitivity S_{I} on that day by analyzing the resulting drop in glucose readings caused by the insulin dosage. These data points (e.g., data 202) are retrieved from continuous glucose monitor 100, memory connected to the display device 202, or user input on the display device in the case of manually received data 202. This S_{I} can then be used to update the patient's overall S_{I}. In some embodiments, sensitivity update algorithm 214 will calculate multiple S_{I} values from different historical time periods, and then combine those values to update the patient's overall S_{I}. For example, the patient's S_{I} can be based on a weighted average of the values over a predetermined period of time, for example, over the past 7 days. This can improve the accuracy of the overall S_{I} while still allowing the system to adapt to the patient's changing physiological condition.

The discussion above regarding therapy algorithm 200 has assumed that glucose is the relevant analyte to be modeled. However, any suitable analyte that has a response to medication can also be modeled in a similar fashion. For example, analytes that can be used as the basis for therapy algorithm 200 can include glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, and uric acid.

An optimization algorithm 220 can then be used to determine an optimized therapy using the revised model of therapy algorithm 200. Like therapy algorithm 200, optimization algorithm 220 is operated on display device or a suitable connected computing device. Optimization algorithm 220 retrieves the results of therapy algorithm 200 (updated glucose lowering curve 210) from a connected memory as the basis for its operation. The optimized dosage determination can be accomplished by using glucose lowering curve 210 to determine the resulting glucose response for each of a set of alternative medication dosages. For example, in some embodiments, optimization algorithm 220 will perform a selection step 222 to select a set of alternate dosages for analysis. The alternate dosages may be based on a percentage of the existing medication dose. The alternate dosages may be selected within a predefined range of the existing dose, such as +/−20% of the original dose. For example, the set of alternate medication dosages may include 80%, 85%, 90%, 95%, 105%, 110%, 115%, and 120% of the existing glucose dose 223. This set of dosages is then combined with glucose lowering curve 210 in a glucose response step 224 that produces the predicted glucose response for each of the alternate medication dosages based on the updated glucose lowering curve 210. Each of the updated glucose lowering curve 210 may have different peak glucose value as determined by the insulin sensitivity S_{I} and the corresponding dose associated with the percentage dose change. The user's glucose response to a current medication dose may be superimposed with a glucose lowering curve for the dose amount that corresponds to the percent increase or decrease from the current medication dose to the alternate medication dosage. For example, if the user administered a 10 U dose of basal insulin at the start of a 24 hour window, and 24 hours of glucose data is collected, the impact of switching to a 12 U dose can be determined by the glucose lowering impact of 2 U of insulin based on the glucose lowering curve.

The resulting predicted glucose responses for each of the alternate medication dosages are analyzed by an analysis algorithm 230 that can determine a range of metrics that describe the data. For example, analysis algorithm 230 may determine one or more glucose metrics. The glucose metrics may include at least one of median glucose versus a target glucose, time in a predetermined glucose range (TIR), time in hypoglycemia (e.g., time below range (TBR), a number of hypoglycemic events, glycemic variability, and other metrics. These metrics can then be compared across each of the alternate medication dosages to determine which dosage optimized the glucose metric or metrics. In this way, optimization algorithm 220 can perform an output step 240 to present a recommendation for an optimized dosage of insulin based in a predictive manner based on historical data. For example, if the glucose metric is time in range, the alternate medication dosage that produced the highest time in range may be selected as the recommended medication dose. Alternatively, if the glucose metric is the number of hypoglycemic events, any medication dosage that results in a number of hypoglycemic events may be excluded or otherwise ranked lower in the analysis. Alternatively, if the glucose metrics are time in range and median glucose vs. target, the analysis may select a recommended dose that achieves a time in range above a certain minimum time in range, and among doses satisfying that criteria, will further select a recommended dosage based on the dose that provided the glucose median closest to the target median. Metrics may also be used to sequentially filter the results. For example, a minimum time in range may be used to filter the results, and then the optimum result may be selected by application of a second metric, such as median glucose vs. target. In some embodiments, metrics may also have different analytical weights. Thus, for example, the number of hypoglycemic events may be given a higher weight versus the median glucose vs. target, meaning that a result with fewer hypoglycemic events but a worse median glucose vs. target would be selected instead of a result with many hypoglycemic events but a good median glucose vs. target. In some embodiments, the selection metrics may be changed based on information related to the patient or inputs from the health care professional. For example, a patient with generally greater variability in daily glucose levels may be assigned metrics that give greater weight to a lower number of hypoglycemic events. This differs from existing titration algorithms that are reactive in nature because they recommend a dosage chain when glucose readings exceed certain bounds. The analysis may be based on a mathematical model.

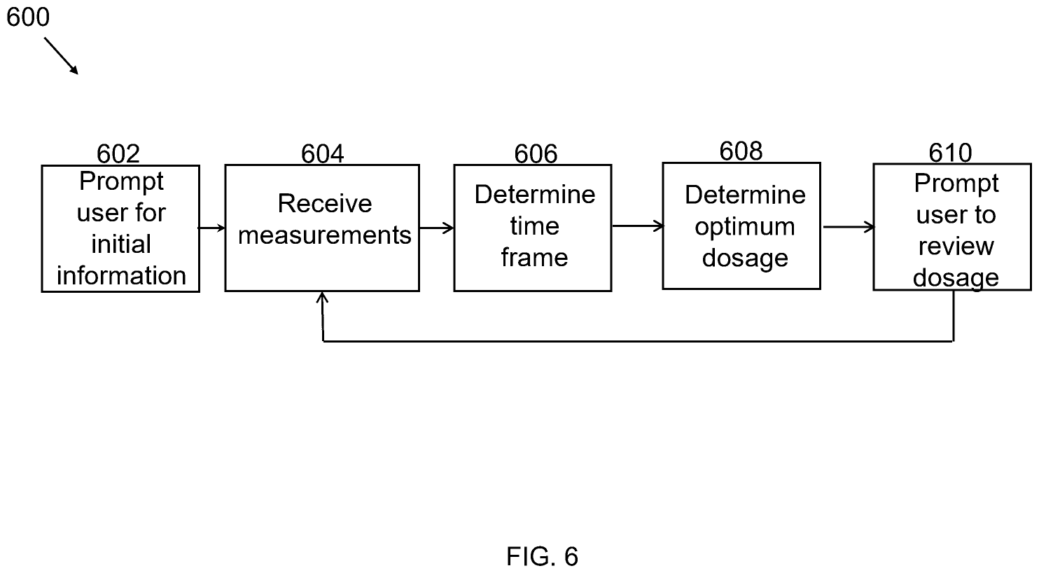

As shown in FIG. 6, a method 600 of using therapy algorithm 200 and optimization algorithm 220 begins with a prompt 602 that requests initial information from the user. Here, therapy algorithm 200 receives information about the user, such as physiological data, including weight and age, and medication data, including daily insulin dosages. This information can be received by manual input from the patient or health care professional using as suitable device, such as reader device 120 or local computer system 170. Alternatively, therapy algorithm 200 may automatically retrieve this information from a suitable memory located either locally or remotely through a data network, such as from an electronic medical record.

Next, in a measurements step 604 therapy algorithm 200 can then either receive glucose data from continuous glucose monitor 100. Therapy algorithm 200 may receive glucose data from memory, or from a remote computer or server.

At a step 606, therapy algorithm 200 determines the time frame used for this analysis. The time frame can be automatically selected by therapy algorithm 200, or can be entered by a user. For example, the display device may display a prompt for a user to enter or to select a period of time for the analysis, such as 7 days, 10 days, 14 days, among other periods of time.

In a step 608, optimization algorithm 220 can then proceed to calculate a recommended dosage based on the user's existing insulin dosage and glucose lowering curve 210 as discussed above. The dosage variation parameters may be automatically selected, or may be adjusted by the user.

Finally, in a step 610, the user can be prompted to review the recommended dosage on the display device. The user can then accept the dosage or adjust the dosage as needed.

In step 610, the recommended dosage can be displayed and used in several different ways. For example, step 610 can include a prompt that is shown to the user informing them that an optimized dosage is available. In some embodiments, reader device 120 can receive the recommended dosage. In some embodiments, reader device 120 is a computing device or mobile device of the patient, and the patient can review the recommended dosage. In some embodiments, the health care professional has a reader device 120 in the form of a computing device associated with the health care professional, who can review the recommended dosage and determine whether to modify treatment. In these embodiments, the patient may receive a copy of the recommended dosage, or may only receive a notification that the recommended dosage is available. Other data may also be included with the recommended dosage, for example, including the data received from continuous glucose monitor 100 and glucose lowering curve 210.

The user may make a selection on the display device to accept the dosage, and a medication delivery device in communication with the display device may automatically deliver the dose (such as an infusion pump). Alternately, the user may confirm the dose and manually administer the medication, such as with an injection pen. The administration of the dose may be recorded by a dose monitor of the injection pen and transmitted to the display device. As discussed above and as shown in FIG. 6, after step 610 the process begins again at step 604 to continuously optimize the dosage based on new glucose information.

Therapy algorithm 200 and optimization algorithm 220 can be used for any type of glucose medication that results in a response that can be modeled as glucose lowering curve 210. For example, therapies may include long-lasting or basal insulin, fast-acting insulin, GLP-1RA, metformin, sulfonylureas, DPP4 inhibits, and other medications.

As discussed above, algorithms such as therapy algorithm 200 and optimization algorithm 220 benefit from access to a patient's medical history. Specifically, these types of algorithms can provide improved analysis and optimization results if the patient's current medication therapies are available. Manual entry of therapies by a patient or health care professional can introduce errors and is time consuming. Thus, automated access and uploading of medical therapies is preferred to improve accuracy and reduce burdens on system users. Therefore, there is a need for methods and systems for automatically accessing a patient's medical therapy information for use in glucose monitoring and treatment systems.

Embodiments of the present disclosure access a patient's current medical therapies by using an application programming interface (“API”) to receive information from a patient's pharmacy records from a pharmacy information system (“PIS”). In some embodiments, the information needed to access the PIS, which can include an account or identification number unique to the patient, is automatically imported from an existing prescription or medication label through a text recognition process using an existing camera on a mobile device of a user. The system then uses the API to request updated information regarding the patient's medical therapies. These embodiments have the benefit of reducing errors caused by manual entry of medications and also reduces burden on the patient or health care professional.

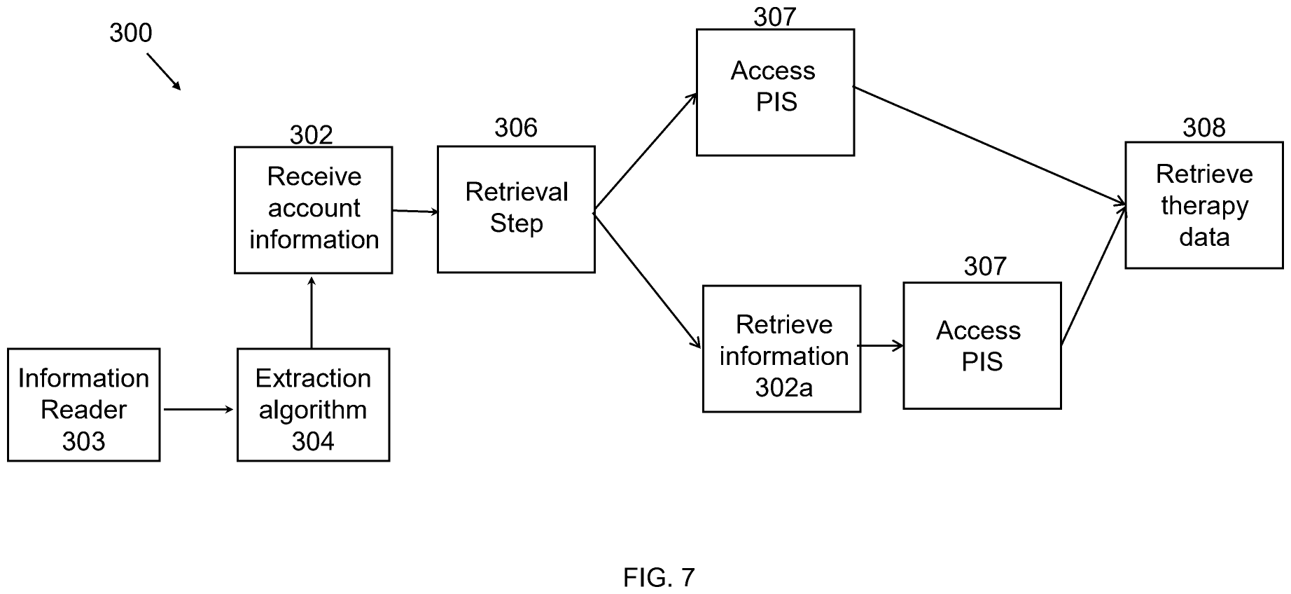

The embodiments discussed above and below can operate on any suitable computing device. For example, the embodiments may be run as a program or application on reader device 120. In an embodiment, as illustrated in FIG. 7, an access algorithm 300 first receives account information 302, which is information unique to a patient's PIS account. For example, account information 302 can be an account number, a unique identifier, or any other suitable combination of information. In some embodiments, account information 302 is manually entered by the patient or health care professional using suitable means such as a keyboard associated with the computing device. In other embodiments, account information 302 is automatically entered by an reader device 303. Reader device 303 can be any device that is linked to the user's computing device that can import account information 302. For example, reader device 303 may be a camera that can take images of physical objects. In these embodiments, the user may use the camera to take an image of an object or document that includes account information 302, such as a prescription form or a medication package. Reader device 303 can transmit that image to an account information analyzer 304 that extracts the relevant information from the image using suitable techniques. Examples of these techniques include optical character recognition techniques for text-based information, or techniques that identify data patterns such as bar codes or quick-response codes.

In some embodiments, the information retrieved by reader device 303 or manually entered by the patient may not be directly linked to the patient's PIS, but instead may be account information 302a that uniquely identifies a different electronic medical record (“EMR”) associated with the patient. This may be the case, for example, when a health care professional is supplying information to access algorithm 300 and inputs the patient's EMR account number.

Access algorithm 300 then proceeds to a retrieval step 306 where account information 302 or account information 302a is used to communicate with the PIS to retrieve the patient's therapy and medication information. In embodiments where account information 302 corresponds directly with the PIS, retrieval step 306 involves a direct access to the PIS step 307 by using the relevant API command to request the patient's therapy and medication information in a therapy data retrieval step 308. This API command can be transmitted using a suitable remote data network, such as network 190. In some embodiments, electronic healthcare data may be exchanged between components of the system by a Fast Healthcare Interoperability Resources standard developed by Health Level Seven International (HL7 FHIR).

In other embodiments, retrieval step 306 may instead communicate with an EMR using alternate account information 302a using a suitable API. This communication may then retrieve the account information 302 that corresponds to the patient's PIS. Retrieval step 306 may then proceed as discussed above.

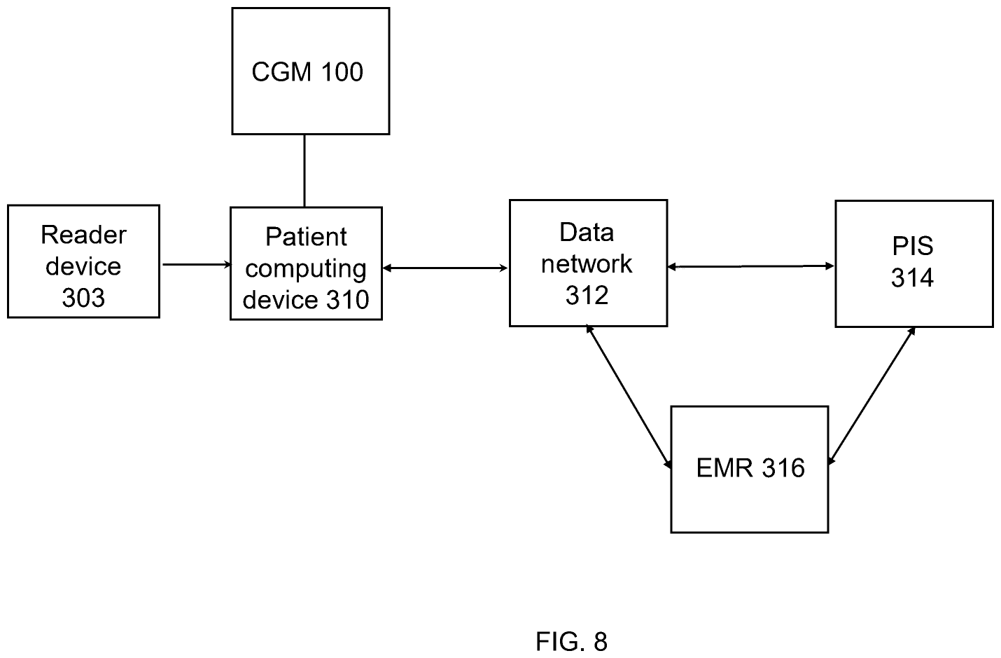

FIG. 8 is a system diagram showing the various databases and networks accessed above. As seen in FIG. 8, a patient computing device 310 is connected to a data network 312. Patient computing device 310 can be any suitable computing device, such as reader device 120 or local computing system 170. Reader device 303 is connected to patient computing device 310. Data network 312 facilitates access to a PIS database 314 an EMR database 316 for the operations discussed above. Data network 312 can be any suitable wireless or wired data network. Continuous glucose monitor (“CGM”) 100 is also shown as being connected to patient computing device 310.

The display device may include an analyte monitoring software application that generates and displays analyte data and analyte data reports to the user based on data collected by an analyte monitoring sensor in communication with the display device. The software application may generate and provide recommendations for maintaining analyte levels in a target range or achieving other goals, such as recommendations to adjust medication dosages or to engage in self-care behavior. The analyte monitoring software application may receive the updated medication information from the PIS or EMR as described herein. The software application may then make more specific recommendations tailored to the particular medications taken by the user, such as to increase or decrease a dose of the medication, to add a second medication to the therapy regimen, or to adjust self-care behaviors, such as exercise, dieting, weight management, and sleep, among others.

Advantages of these systems and methods include the inclusion of a patient's medication therapy in glucose analysis and optimization systems, such as therapy algorithm 200. The automated nature of access algorithm 300 reduces errors and burden on patients and health care professionals when providing the medication therapy information. The use of an API to access the EMR and PIS also improves security because direct access to these databases is not required because the API involves only sending a specified set of requests to the database, not direct interaction with the database.

Algorithms such as therapy algorithm 200 discussed above will analyze glucose data and provide individualized recommendations for dosage adjustments based on the patient's glucose levels. The medications dosages being changed are subject to limitations in terms of both maximum authorized dosages and limitations related to factors such as the patient's individual history, physiological condition, and other medical therapies. Because these algorithms provide dosage updates frequently, there exists a need to allow for oversight of dosage recommendations by a health care professional in near real-time and remotely to avoid the burden of adding clinical visits that burden both the patient and health care professional.

Embodiments of the present disclosure include systems and methods of establishing limits on recommended medication dosages from titration algorithms that analyze glucose data and provide individualized recommendations for dosage adjustments based on the patient's glucose levels. The health care professional can remotely update a range of limitations that can be implemented by the algorithm. The health care professional is notified if these limitations are met or approached so they can take suitable action, such as updating the limitation or contacting the patient. This allows the titration algorithm to operate continuously to optimize dosages while maintaining the customized oversight needed to improve a patient's treatment.

In an embodiment, a dosage algorithm may include one or more limitations 400 that constrain the dosages that the algorithm can recommend. Limitations 400 can be one or more limits related to medication dosage. For example, limitations 400 can be a maximum dosage for any given therapy, such a total daily basal insulin dose or a single rapid acting dose. These maximum dosages can be tailored to each specific medication. For example, the total basal dosage may be have one limit, while the breakfast rapid acting dose may have a separate limit. For a user taking a rapid acting dose at each meal, there may be a different limit set for the maximum dose for each meal. These limits may be expressed in various ways, such as a maximum amount of medication, or as a ratio or percentage of an initial dose. For example, the recommended dose is no greater than a set percentage of an initial dose amount, e.g., the recommended dose is no greater than 50% of the initial dose. In another example, the limit may be expressed as a ratio or percentage of another limit. For example, the total rapid acting doses may be limited to a percent of the total basal insulin dose. Limitations 400 may also be set to consider combined dosages, for example the combined insulin dosage recommended for the patient across all meals.

In some embodiments, these limits are applied to the typical or “fixed” portion of the meal-time doses. That is, the automatic titration system is limited in how much it can increase this typical portion of the recommended dose. The dose recommendation made to the patient is made up of this fixed portion (which may be different for each meal—e.g., breakfast, lunch and dinner) plus other portions, such as insulin for bringing down high glucose (also known as the correction portion). The limit in this embodiment is on the fixed portion only. In alternative embodiments, this limit may be applied to the entire recommendation made to the patient.

Limits may also be set on other insulin regimen parameters, such as carbohydrate ratios sometimes used to calculate insulin doses for meals, and insulin sensitivity factors (or correction factors) used to calculate the portion of the dose needed to alter the patient's glucose level. These limits may be set individually or as a single percentage limit that is applied to all or some subset of these regimen parameters.

Limitations 400 can also be set to change depending on conditions specific to the patient. For example, a limit of a maximum dosage for a given rapid acting insulin dose may be set to have a first value if the total insulin dose is less than a first threshold, but may be set to a different, second value if the total insulin dose exceeds the first threshold. In this way, limits can be personalized and tuned to a patient's specific needs or a health care provider's desired level of oversight.

In some embodiments the system could recommend limitations 400 for the health care professional. In some embodiments, these recommended limitations 400 can be predetermined. In other embodiments, they may be based on the patient's current medication history and data from continuous glucose monitor 100. This can allow for customized recommendations that are more closely tailored to the patient. For example, the limits may be based on one or more glucose metrics. The glucose metrics may include a measure of central tendency, such as a glucose median, or an average glucose, among others. For example, if a patient's median glucose is elevated far above a preset goal and the user has low glucose variability, the system could recommend higher limitations 400 on medication dosage than a situation where the glucose median is closer to the preset goal. This is because a higher median glucose indicates that the user is further from optimal control and thus could accommodate higher dose increases relative to a user whose glucose median is closer to the preset goal. These recommendations can be presented both initially, when the health care professional is setting the initial limitations 400, and also at regular intervals of time. In some embodiments, the recommendations can also be triggered by limitations 400 being met or exceeded. The system can analyze the specific limit or limits in limitations 400 that were exceeded and present a tailored recommendation to the health care professional as discussed above.

In some embodiments, the system includes a user interface that includes a first screen for the health care professional to set initial medication regimen parameters. For example, the health care professional may set a dosage for each medication, such as a long-acting insulin dose and one or more rapid acting dose amounts. The user interface may present a second screen in which the HCP may set a limit for each of the doses, or for a combination of the doses, i.e., a maximum total insulin amount.

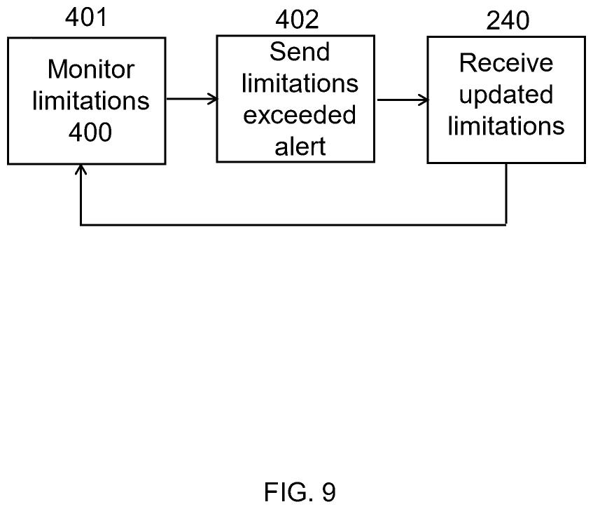

As shown in FIG. 9, limitations 400 are actively monitored in a monitoring step 401, and once the limitations 400 are met or exceeded, the system is programmed to remotely alert the health care professional in an alert step 402 using a suitable network, such as data network 190. The health care professional will be alerted to review the recommendation and update limitations 400 in an update step 404 if needed. This alert, and access to limitations 400, can be implemented on any suitable computing device, such as a computer of mobile device of a health care professional.

While the discussion above relates primarily to insulin-based medical therapies, any relevant therapy subject to an automated dosage algorithm can implement limitations 400 as discussed here.

Medication dose guidance applications 1000 (hereafter “applications 1000”), such as the applications corresponding to therapy algorithm 200 above, can provide improved dosage optimization that improves patients outcomes. These applications are operated by a computing device that is typically associated with the patient, such as reader device 120, local computing system 170, continuous glucose monitor 100, or insulin delivery devices such as insulin delivery pens.

For safety and security purposes, applications 1000 must be initialized prior to beginning operation. This ensures that application 1000 is properly configured and is being assigned to a patient under the supervision of a health care professional. Part of this initialization process is the input of a code or sequence 1002 that unlocks or initializes application 1000 for use. This code 1002 is typically provided by the manufacturer of application 1000 to the health care provider, who must manually input the code into application 1000 using the relevant computing device. Access to this code is usually accomplished through a call or other communication between the health care professional and the manufacturer, which is burdensome and difficult to coordinate with the patient's clinical visit. Thus, there exists a need to streamline the process of initializing applications 1000 to reduce burden on the health care professional.

Embodiments of the present disclosure address these issues by integrating code 1002 into the health care professional's existing electronic medical record 1004. Code 1002 is therefore easily accessible using existing systems that the health care professional is already familiar with during the clinical visit for initializing application 1000. Additional benefits of the embodiments discussed below include storage of code 1002 in a secure database (electronic medical record 1004) associated with the patient for any potential future use.

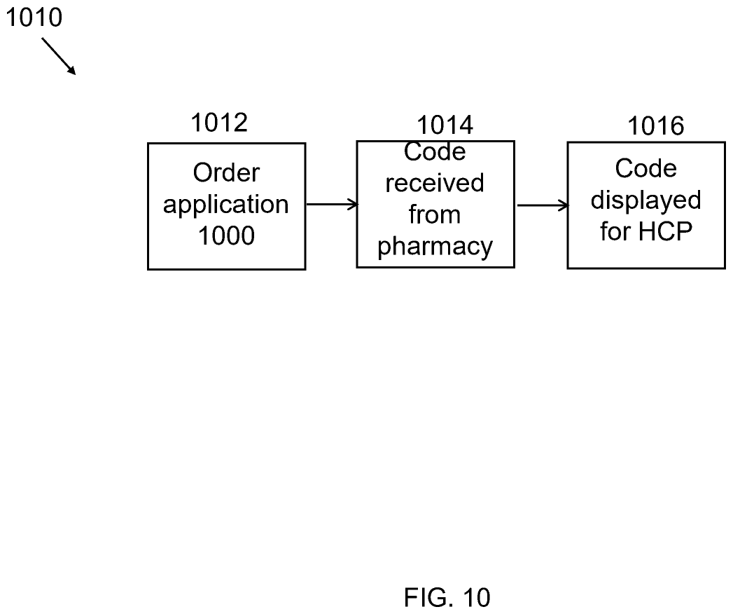

As shown in FIG. 10, a method 1010 of initializing application 1000 begins with an order step 1012. The health care professional places an order for a dose guidance system. In some embodiments, the dose guidance system is only a copy of application 1000 that will be installed on a suitable computing device, such as reader device 120 that is already in the patient's possession. In other embodiments, the order may include one or more of application 1000, a computing device, such as a connected medication delivery device, such as an injection pen, medication, such as insulin, or a glucose monitoring device, such as a CGM. In these embodiments application 1000 may be pre-installed on the computing device. The pharmacy retrieves a code specific to the particular instance of the dose guidance app and/or the associated connected medication delivery device (e.g., insulin delivery pen). The code may be displayed on a packaging label of the medication delivery device. Alternately, a database or table stored in a computing device of the pharmacy includes a list of codes and associated serial numbers for the dose guidance system. The codes may be alphanumeric codes. Alternately, a single code may be used to enable multiple dose guidance systems of the same type. The pharmacy computing device may include an authentication system that stores device serial numbers and their associated codes. The authentication system may be a web server accessible by the pharmacy computing device and by the dose guidance application. When an order is received, the pharmacy enters the serial number and the authentication system returns the associated authentication cod.

In some embodiments, the authentication system stores HCP identifier information, and generates a code that is specific to each HCP. The HCP identifier and authentication code are needed to initialize the application. The application may communicate with the authentication system to determine if the entered HCP identifier and authentication code match the stored information in the authentication system.

The pharmacy computing device communicates the authentication code 1002 to electronic medical record 1004 in a step 1014. This can occur in one of several ways as discussed above. For example, code 1002 may be physically printed on packing or documentation associated with the dose guidance system. The pharmacy will then manually upload code 1002 into electronic medical record 1004. In other examples, the pharmacy may have an authentication system that automatically produces code 1002 for the computing device having application 1000. This can be triggered by the placement of the order by the pharmacy, or may be triggered by an additional action by the pharmacy, such as scanning a barcode of the received computing device or taking other similar actions to acknowledge receipt of the computing device. Once code 1002 is received in this way, it can be uploaded to electronic medical record 1004. In any of these embodiments, code 1002 may be a code unique to application 1000, or may be a more general code that covers several versions or implementations of application 1000.

In a step 1016, code 1002 is displayed to the health care professional from electronic medical record 1004 for upload into application 1000. This can occur at the clinical visit in real-time. The uploading process can be accomplished by manual entry of code 1002 into application via a suitable input device, such as a keyboard of the computing device. In other embodiments, the uploading process can be automated by application 1000 using a suitable reader device 1006 to read code 1002. For example, code 1002 may be displayed in electronic medical record 1004 as a bar code. The health care professional can retrieve code 1002 and display it on a suitable screen, and reader device 1006 can be used to scan bar code 1002 by application 1000.

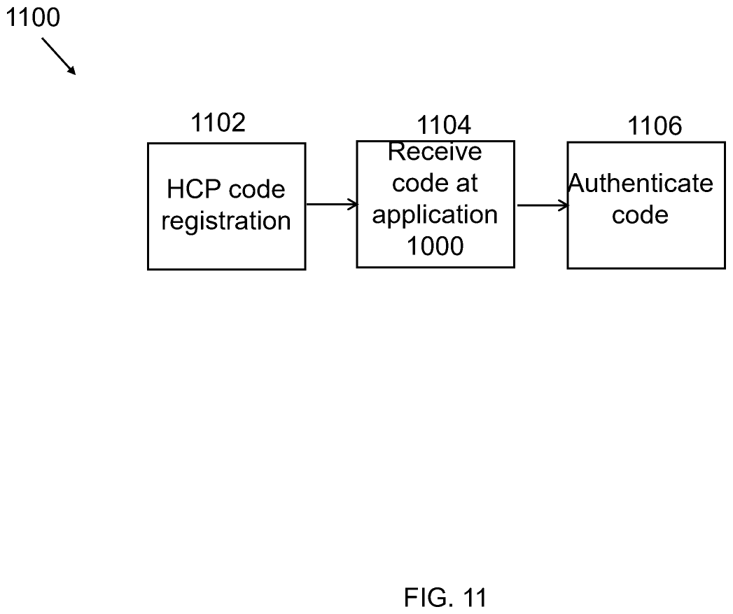

In other embodiments, as shown in FIG. 11, code 1002 is not associated specifically with application 1000, but is instead associated with the health care professional. In these embodiments, in a method 1100 the health care professional must perform a one-time code registration step 1102 where they register a code 1002 with the relevant manufacturer of application 1000. This code 1002 is saved by the health care professional in any suitable manner, such as in electronic medical record 1004. At a clinical visit, the health care professional application 1000 then receives the health care professional's code 1002 in an input step 1104. This input step 1104 can either involve manual input as discussed above, or input via reader device 1006 as discussed above. Application 1000 then performs an authentication step 1106 where application 1000 verifies code 1002. This authentication step can be performed by using a suitable data network to contact a remote computing device, such as a server of the manufacturer.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

Exemplary embodiments are set out in the following numbered clauses:
1. A method of determining an optimized medication dosage for a glucose disorder, the method comprising:
   receiving, by one or more processors, glucose data from a continuous glucose monitor worn by a patient;
   receiving, by the one or more processors, medication data for the patient;
   selecting, by the one or more processors, a set of alternate medication dosages based on the medication data;
   determining a predicted glucose response for each medication dosage of the set of alternate medication dosages;
   analyzing each predicted glucose response to determine an optimized medication dosage of the set of medication dosages; and
   outputting the recommended medication dosage by a display device in communication with the one or more processors.
2. The method of clause 1, wherein the glucose data comprises glucose measurements recorded on a plurality of days.
3. The method of clause 1 or 2, wherein the predicted glucose response for each medication dosage is determined based in part on the glucose data and a glucose lowering curve.
4. The method of clause 1, 2 or 3, wherein the updating further comprises updating an insulin sensitivity for the patient using the glucose data and the medication data.
5. The method of any preceding clause, wherein the updating further comprises updating a peak insulin response for the patient using the glucose data and the medication data.
6. The method of any preceding clause, wherein the medication data includes a treatment dosage and a treatment time corresponding to when the treatment dosage was administered, and
   wherein updating the glucose lowering curve further comprises determining an insulin sensitivity of the patient by analyzing a response in glucose measurements in the glucose data after the treatment time.
7. The method of any preceding clause, wherein the glucose data comprises glucose measurements recorded on a plurality of days,
   wherein the insulin sensitivity is one of a plurality of insulin sensitivities, each insulin sensitivity corresponding to each day of the plurality of days, and
   wherein updating the glucose lowering curve further comprises taking an average of the plurality of insulin sensitivities.
8. The method of any preceding clause, wherein the selecting the set of alternate medication dosages further comprises determining a new dosage by modifying an existing dosage from the medication data by at least one of increasing or decreasing the existing dosage.
9. The method of any preceding clause, wherein analyzing each predicted glucose response further comprises calculating a metric for each predicted glucose response, the metric being at least one of median glucose versus a target glucose, time in a predetermined glucose range, time in hypoglycemia, number of hypoglycemic events, or glycemic variability.
10. The method of any preceding clause, further comprising receiving a limitation on a medication dosage from a remote computing device, wherein selecting the set of alternate medication dosages is based in part on the limitation.
11. The method of clause 10, wherein the limitation comprises one or more of a maximum dosage for an individual medication; a maximum total dosage of medication for a predetermined time period, and a maximum dosage for an individual medication dosage related to a second medication dosage.
12. The method of any preceding clause, wherein the receiving medication data further comprises:
   receiving account information for an account associated with the patient receiving medication, the account having information related to the medication data; and
   using the account information to access the account to retrieve the information related to the medication data.
13. The method of clause 12, wherein receiving account information further comprises acquiring the account information from an object using a reader device.
14. The method of clause 13, wherein the reader device comprises a camera that is used to acquire the account information from the object.
15. The method of clause 12, 13, or 14, wherein the account is an electronic medical record, and wherein the information related to the medication data is a second account information for a second account that comprises the medication data, the method further using the second account information to access the second account to retrieve the medication data.
16. The method of any of clauses 12 to 15, wherein the information related to the medication data is incorporated into the recommended medication dosage.
17. The method of any preceding clause, wherein the medication data is received from an insulin delivery device.
18. The method of any preceding clause, wherein the medication data is received from a user input.
19. The method of any preceding clause, wherein the medication data comprises an insulin data.
20. A method of initializing an application comprising a medication dosage guidance algorithm on a display device of a patient, the method comprising:
   transmitting from a computing device of a health care provider (HCP), to a pharmacy computing device, an order for the application;
   receiving, from the pharmacy information system, a code for initializing the application at an electronic medical record of a health care professional using an authentication system;
   displaying the code using a display device in communication with the electronic medical record; and
   receiving the code by the display device of the patient to initialize the application.
21. The method of clause 20, wherein the receiving the code further comprises:
   receiving an inquiry at an authentication system for the code from the pharmacy; and
   transmitting the code to the pharmacy in response to the inquiry.
22. The method of clause 20 or 21, wherein receiving the code at the display device comprises scanning the code by a reader device of the display device.
23. The method of clause 20, 21 or 22, wherein the authentication system receives an identifier of the HCP and returns an authentication code based in part on the identifier of the HCP.
24. A system for determining an optimized therapy for a glucose disorder, the system comprising:
   a continuous glucose monitor, comprising:
   a glucose sensor comprising a first portion configured to be arranged above the skin and a second portion configured to be arranged below the skin and in contact with interstitial fluid to sense glucose within the interstitial fluid, and
   sensor electronics coupled to the glucose sensor and comprising a memory, communication circuitry, and a processor coupled to the memory and communication circuitry; and
   a computing device including a second processor, a second memory, and a display, wherein the computing device is in communication with the continuous glucose monitor,
wherein the second memory stores instructions that when executed by the second processor cause the second processor to:
   receive glucose data from the continuous glucose monitor;
   receive medication therapy data for the patient at the computing device;
   select a set of medication therapy dosages based on the medication therapy data;
   determine a predicted glucose response for each medication therapy dosage of the set of medication therapy dosages;
   analyze each predicted glucose response to determine an optimized medication therapy dosage of the set of medication therapy dosages; and
   output the optimized medication therapy dosage on the display of the computing device.
25. The system of clause 24, wherein the glucose data comprises glucose measurements recorded on a plurality of days.
26. The method of clause 24 or 25, wherein the predicted glucose response for each medication therapy dosage is determined based in part on the glucose data and a glucose lowering curve.
27. The method of any of clauses 24 to 26, wherein the updating includes instructions that further cause the second processor to update an insulin sensitivity for the patient using the glucose data and the medication therapy data.
28. The method of any of clauses 24 to 27, wherein the updating includes instructions that further cause the second processor to update a peak insulin response for the patient using the glucose data and the medication therapy data.
29. The system of any of clauses 24 to 28, wherein the medication therapy data includes a treatment dosage and a treatment time corresponding to when the treatment dosage was administered, and
   wherein the instructions to update the glucose lowering curve cause the second processor to determine an insulin sensitivity of the patient by analyzing a response in glucose measurements in the glucose data after the treatment time.
30. The system of any of clauses 24 to 29, wherein the glucose data comprises glucose measurements recorded on a plurality of days,
   wherein the insulin sensitivity is one of a plurality of insulin sensitivities, each insulin sensitivity corresponding to each day of the plurality of days, and
   wherein the instructions to update the glucose lowering curve further cause the second processor to take an average of the plurality of insulin sensitivities.
31. The system of any of clauses 24 to 30, wherein the instructions to select the set of medication therapy dosages further cause the second processor to determine a new dosage by modifying an existing dosage from the medication therapy data by at least one of increasing or decreasing the existing dosage.
32. The system of any of clauses 24 to 31, wherein the instructions to analyze each glucose response further cause the second processor to calculate a metric for each glucose response, the metric being at least one of median glucose versus a target glucose, time in a predetermined glucose range, time in hypoglycemia, number of hypoglycemic events, and glycemic variability.
33. The system of any of clauses 24 to 32, wherein the instructions further cause the second processor to receive a limitation on a medication therapy dosage from a remote computing device, wherein the selecting a set of medication therapy dosages is based in part on the limitation.
34. The system of clause 33, wherein the limitation is selected from the group consisting of a maximum dosage for an individual medication; a maximum total dosage of medication for a predetermined time period, and a maximum dosage for an individual medication dosage related to a second medication dosage.
35. The system of any of clauses 24 to 36, wherein the instructions to receive medication therapy data further cause the second processor to:
   receive account information for an account associated with the patient receiving medication therapy, the account having information related to the medication therapy data; and
   use the account information to access the account to retrieve the information related to the medication therapy data.
36. The system of clause 35, further comprising a reader device, wherein the instructions to receive account information further cause the second processor to use the reader device to acquire the account information from an object.
37. The system of clause 36 or 37, wherein the reader device comprises a camera that is used to acquire the account information from the object.
38. The system of clause 36, 37 or 38, wherein the account is an electronic medical record, and wherein the information related to the medication therapy data is a second account information for a second account that comprises the medication therapy data, the instructions further comprising instructions to cause the second processor to use the second account information to access the second account to retrieve the medication therapy data.
39. The system of any of clauses 36 to 39, wherein the information related to the medication therapy data is incorporated into the recommended medication dosage.
40. The system of any of clauses 24 to 40, wherein the medication therapy data is received from an insulin delivery device.
41. The system of any of clauses 24 to 41, wherein the medication therapy data is received from a user input.
42. The system of any of clauses 24 to 42, wherein the medication therapy data comprise an insulin medication therapy.
43. A system for determining an optimized therapy for a glucose disorder, the system comprising:
   a continuous glucose monitor, comprising:
   a glucose sensor comprising a first portion configured to be arranged above the skin and a second portion configured to be arranged below the skin and in contact with interstitial fluid to sense glucose within the interstitial fluid, and
   sensor electronics coupled to the glucose sensor and comprising a memory, communication circuitry, and a processor coupled to the memory and communication circuitry; and
   a computing device including a second processor, a second memory, and a display, wherein the computing device is in communication with the continuous glucose monitor,
wherein the second memory stores instructions that when executed by the second processor cause the second processor to:
   transmit from a computing device of a health care provider, to a pharmacy computing device, an order for the application;
   receive, from the pharmacy information system, a code for initializing the application at an electronic medical record of a health care professional using an authentication system;
   display the code using a display device in communication with the electronic medical record; and
   receive the code by the display device of the patient to initialize the application.
44. The system of clause 43, wherein the receiving the code further comprises instructions that further cause the second processor to:
   receive an inquiry at an authentication system for the code from the pharmacy; and
   transmit the code to the pharmacy in response to the inquiry.
45. The system of clause 44 or 45, wherein receiving the code further comprises instructions that cause the second processor to scan the code by a reader device of the display device.
46. A system for determining an optimized therapy for a glucose disorder, the system comprising:
   a continuous glucose monitor, comprising:
   a glucose sensor comprising a first portion configured to be arranged above the skin and a second portion configured to be arranged below the skin and in contact with interstitial fluid to sense glucose within the interstitial fluid, and
   sensor electronics coupled to the glucose sensor and comprising a memory, communication circuitry, and a processor coupled to the memory and communication circuitry; and
   a computing device including a second processor, a second memory, and a display, wherein the computing device is in communication with the continuous glucose monitor,
wherein the second memory stores instructions that when executed by the second processor cause the second processor to:
   generate a unique code for a health care provider based on an inquiry from the health care provider;
   receive the unique code at the application; and
   authenticate the unique code to initialize the application.

What is claimed is:

1. A method of determining an optimized medication dosage for a glucose disorder, the method comprising:
receiving, by one or more processors, glucose data from a continuous glucose monitor worn by a patient;
receiving, by the one or more processors, medication data for the patient;
selecting, by the one or more processors, a set of alternate medication dosages based on the medication data;
determining a predicted glucose response for each medication dosage of the set of alternate medication dosages;
analyzing each predicted glucose response to determine an optimized medication dosage of the set of medication dosages; and
outputting the optimized medication dosage by a display device in communication with the one or more processors.

2. The method of claim 1, wherein the glucose data comprises glucose measurements recorded on a plurality of days.

3. The method of claim 1, wherein the predicted glucose response for each medication dosage is determined based in part on the glucose data and a glucose lowering curve.

4. The method of claim 3, wherein the determining an optimized medication dosage further comprises updating an insulin sensitivity for the patient using the glucose data and the medication data.

5. The method of claim 1, wherein the determining an optimized medication dosage further comprises updating a peak insulin response for the patient using the glucose data and the medication data.

6. The method of claim 3, wherein the medication data comprises a treatment dosage and a treatment time corresponding to when the treatment dosage was administered,
the method further comprising updating the glucose lowering curve by determining an insulin sensitivity of the patient by analyzing a response in glucose measurements in the glucose data after the treatment time.

7. The method of claim 6, wherein the glucose data comprises glucose measurements recorded on a plurality of days,
wherein the insulin sensitivity is one of a plurality of insulin sensitivities, each insulin sensitivity corresponding to each day of the plurality of days, the method further comprising updating the glucose lowering curve by taking an average of the plurality of insulin sensitivities.

8. The method of claim 1, wherein the selecting the set of alternate medication dosages further comprises determining a new dosage by modifying an existing dosage from the medication data by at least one of increasing or decreasing the existing dosage.

9. The method of claim 8, wherein analyzing each predicted glucose response further comprises calculating a metric for each predicted glucose response, the metric being at least one of median glucose versus a target glucose, time in a predetermined glucose range, time in hypoglycemia, number of hypoglycemic events, or glycemic variability.

10. The method of claim 1, further comprising receiving, by the one or more processors, a limitation on a medication dosage from a remote computing device, wherein selecting the set of alternate medication dosages is based in part on the limitation.

11. A system for determining an optimized therapy for a glucose disorder, the system comprising:
a continuous glucose monitor, comprising:
a glucose sensor comprising a first portion configured to be arranged above the skin and a second portion configured to be arranged below the skin and in contact with interstitial fluid to sense glucose within the interstitial fluid, and
sensor electronics coupled to the glucose sensor and comprising a memory, communication circuitry, and a processor coupled to the memory and communication circuitry; and
a computing device comprising a second processor, a second memory, and a display, wherein the computing device is in communication with the continuous glucose monitor,
wherein the second memory stores instructions that when executed by the second processor cause the second processor to:
receive glucose data from the continuous glucose monitor;
receive medication therapy data for the patient at the computing device;
select a set of medication therapy dosages based on the medication therapy data;
determine a predicted glucose response for each medication therapy dosage of the set of medication therapy dosages;
analyze each predicted glucose response to determine an optimized medication therapy dosage of the set of medication therapy dosages; and
output the optimized medication therapy dosage on the display of the computing device.

12. The system of claim 11, wherein the glucose data comprises glucose measurements recorded on a plurality of days.

13. The method of claim 11, wherein the predicted glucose response for each medication therapy dosage is determined based in part on the glucose data and a glucose lowering curve.

14. The method of claim 13, wherein the determining an optimized medication dosage comprises instructions that further cause the second processor to update an insulin sensitivity for the patient using the glucose data and the medication therapy data.

15. The system of claim 13, wherein the medication therapy data comprises a treatment dosage and a treatment time corresponding to when the treatment dosage was administered, and wherein the instructions further cause the second processor to update the glucose lowering curve by determining an insulin sensitivity of the patient by analyzing a response in glucose measurements in the glucose data after the treatment time.

16. The system of claim 15, wherein the glucose data comprises glucose measurements recorded on a plurality of days, wherein the insulin sensitivity is one of a plurality of insulin sensitivities, each insulin sensitivity corresponding to each day of the plurality of days, and wherein the instructions further cause the second processor to update the glucose lowering curve by taking an average of the plurality of insulin sensitivities.

17. The method of claim 11, wherein the determining an optimized medication dosage comprises instructions that further cause the second processor to update a peak insulin response for the patient using the glucose data and the medication therapy data.

18. The system of claim 11, wherein the instructions to select the set of medication therapy dosages further cause the second processor to determine a new dosage by modifying an existing dosage from the medication therapy data by at least one of increasing or decreasing the existing dosage.

19. The system of claim 11, wherein the instructions to analyze each glucose response further cause the second processor to calculate a metric for each glucose response, the metric being at least one of median glucose versus a target glucose, time in a predetermined glucose range, time in hypoglycemia, number of hypoglycemic events, and glycemic variability.

20. The system of claim 11, wherein the instructions further cause the second processor to receive a limitation on a medication therapy dosage from a remote computing device, wherein the selecting a set of medication therapy dosages is based in part on the limitation.
